(19) United States
(12) Patent Application Publication
(10) Pub. No.: US 2023/0292992 A1
(43) Pub. Date: Sep. 21, 2023

(54) Apparatus for Thermally Stable Capsule Endoscope Using Effervescent Formulation for Controlling Balloon Inflation Rate

(71) Applicant: CAPSOVISION, Inc., Saratoga, CA (US)

(72) Inventors: Alexa Nazarian, Boston, MA (US); Phat TRINH, San Jose, CA (US); Mikael TROLLSAS, San Jose, CA (US)

(21) Appl. No.: 17/698,303

(22) Filed: Mar. 18, 2022

(51) Int. Cl. A61B 1/00; A61B 1/04; A61K 9/46

(52) U.S. Cl. CPC A61B 1/00082; A61B 1/041; A61K 9/0007

(57) ABSTRACT

A capsule device with thermally-stable specific gravity control is disclosed. The capsule device comprises a capsule unit adapted to be swallowable by a human subject and an inflatable balloon comprising a thermally-stable effervescent formulation inside the inflatable balloon, where the thermally-stable effervescent formulation has a particular particle size between a first mesh size and a second mesh size, and the inflatable balloon is attached to the capsule unit. After the capsule unit with the inflatable balloon attached is swallowed, the inflatable balloon starts to inflate so as to lower specific gravity of a combination of the capsule unit and the inflatable balloon when the inflatable balloon is exposed to body fluid and the body fluid gets in touch with the thermally-stable effervescent formulation inside the inflatable balloon.

### CROSS REFERENCE TO RELATED APPLICATIONS

The present invention is related to U.S. Pat. No. 10,098,526, granted on Oct. 16, 2018 and PCT Patent Application, Serial No. PCT/US2018/055396, published as WO2020076323A1 on Apr. 16, 2020. The U.S. Patent and PCT Patent Application are hereby incorporated by reference in their entireties.

### FIELD OF THE INVENTION

The present invention relates to diagnostic imaging inside the human body or any other living creature. In particular, the present invention relates to an in-vivo capsule containing effervescent material that is thermally stable up to at least 60° C. and has a shelf-life of at least 12 months.

### BACKGROUND AND RELATED ART

Devices for imaging body cavities or passages in vivo are known in the art and include endoscopes and autonomous encapsulated cameras. Endoscopes are flexible or rigid tubes that pass into the body through an orifice or surgical opening, typically into the esophagus via the mouth or into the colon via the rectum. An image is formed at the distal end using a lens and transmitted to the proximal end, outside the body, either by a lens-relay system or by a coherent fiber-optic bundle. A conceptually similar instrument might record an image electronically at the distal end, for example using a CCD or CMOS sensor array, and transfer the image data as an electrical signal to the proximal end through a cable. Endoscopes allow a physician or a veterinary physician control over the field of view and are well-accepted diagnostic tools. However, they do have a number of limitations, present risks to the patient, are invasive and uncomfortable for the patient, and their cost restricts their application as routine health-screening tools.

Because of the difficulty traversing a convoluted passage, regular endoscopes cannot easily reach the majority of the small intestine. In addition, special techniques and precautions, that add cost, are required to reach the entirety of the colon. Endoscopic risks include the possible perforation of the bodily organs traversed and complications arising from anesthesia. Moreover, a trade-off must be made between patient pain during the procedure and the health risks and post-procedural down time associated with anesthesia.

An alternative in vivo image sensor that addresses many of these problems is the capsule endoscope. A camera is housed in an ingestible capsule, along with a radio transmitter for transmitting data, primarily comprising images recorded by the digital camera, to a base-station receiver or transceiver and data recorder outside the body. The capsule may also include a radio receiver for receiving instructions or other data from a base-station transmitter. Instead of radio-frequency transmission, lower-frequency electromagnetic signals may be used. Power may be supplied inductively from an external inductor to an internal inductor within the capsule or from a battery within the capsule.

An autonomous capsule camera system with on-board data storage was disclosed in the U.S. Pat. No. 7,983,458, entitled “In Vivo Autonomous Camera with On-Board Data Storage or Digital Wireless Transmission in Regulatory Approved Band,” granted on Jul. 19, 2011. This patent describes a capsule system using on-board storage such as semiconductor nonvolatile archival memory to store captured images. After the capsule passes from the body, it is retrieved. Capsule housing is opened and the images stored are transferred to a computer workstation for storage and analysis. For capsule images either received through wireless transmission or retrieved from on-board storage, the images will have to be displayed and examined by diagnostician to identify potential anomalies.

FIG. 1 illustrates an exemplary capsule system with on-board storage. The capsule device 110 includes illuminating system 12 and a camera that includes optical system 14 and image sensor 16. A semiconductor nonvolatile archival memory 20 may be provided to allow the images to be stored and later retrieved at a docking station outside the body, after the capsule is recovered. Capsule device 110 includes battery power supply 24 and an output port 26. Capsule device 110 may be propelled through the gastrointestinal (GI) tract by peristalsis.

Illuminating system 12 may be implemented by LEDs. In FIG. 1, the LEDs are located adjacent to the camera's aperture, although other configurations are possible. The light source may also be provided, for example, behind the aperture. Other light sources, such as laser diodes, may also be used. Alternatively, white light sources or a combination of two or more narrow-wavelength-band sources may also be used. White LEDs are available that may include a blue LED or a violet LED, along with phosphorescent materials that are excited by the LED light to emit light at longer wavelengths. The portion of capsule housing 10 that allows light to pass through may be made from bio-compatible glass or polymer.

Optical system 14, which may include multiple refractive, diffractive, or reflective lens elements, provides an image of the lumen walls (100) on image sensor 16. Image sensor 16 may be provided by charged-coupled devices (CCD) or complementary metal-oxide-semiconductor (CMOS) type devices that convert the received light intensities into corresponding electrical signals. Image sensor 16 may have a monochromatic response or include a color filter array such that a color image may be captured (e.g. using the RGB or CYM representations). The analog signals from image sensor 16 are preferably converted into digital form to allow processing in digital form. Such conversion may be accomplished using an analog-to-digital (A/D) converter, which may be provided inside the sensor (as in the current case), or in another portion inside capsule housing 10. The A/D unit may be provided between image sensor 16 and the rest of the system. LEDs in illuminating system 12 are synchronized with the operations of image sensor 16. Processing module 22 may be used to provide processing required for the system such as image processing and video compression. The processing module may also provide needed system control such as to control the LEDs during image capture operation. The processing module may also be responsible for other functions such as managing image capture and coordinating image retrieval. While FIG. 1 illustrates a capsule endoscope with an archival memory to store captured images, the capsule endoscope may also be equipped with a wireless transmitter to transmit the captures to an external receiver.

After the capsule camera traveled through the GI tract and exits from the body, the capsule camera is retrieved and the images stored in the archival memory are read out through the output port. The received images are usually transferred to a base station for processing and for a diagnostician to examine. The accuracy as well as efficiency of diagnostics is most important. A diagnostician is expected to examine the images and correctly identify any anomaly.

A prevalent challenge of capsule endoscope devices intended for collecting images in the colon is controlling their motion as they travel through the twisty structure of the GI tract. If the capsule Specific Gravity (SG) is less than 1 the device may suspend or float in the liquid in the GI tract such as in the stomach. Thus, on the one hand, it is desirable to have the capsule SG>1 so that the capsule device will pass through the stomach and the small intestine without much difficulty. On the other hand, a capsule device with SG>1 will not easily ascend the ascending colon or may sit stationary in the cecum for a long period of time. For this reason, capsule devices have been designed to traverse the ascending colon by having the capsule device SG become less than 1 by about the time the capsule device reaches the cecum. U.S. Pat. No. 10,098,526 entitled “Capsule Device having Variable Specific Gravity” describes a capsule device with an inflatable balloon unit containing an effervescent material that generates gas when exposed to water. When moisture from the GI tracts diffuses through the balloon membrane, the effervescent material is mobilized and reacts to generate CO₂ (gas), the balloon inflates and the specific gravity of the capsule changes. Embodiments of the capsule include a biodegradable shell that encloses the balloon to prevent moisture from the gastric liquid from contacting the effervescent mixture until the shell has disintegrated. One embodiment, such as that described in U.S. Patent Application Publication, Serial No. 2019/0014977, entitled “Capsule Enteric Coating for Controlling Balloon Expansion Start Time,” consists of a pH dependent, enteric coated shell fixed around the balloon. Once the capsule reaches the higher pH environment of the large intestine, the shell disintegrates, and the balloon unit inflates, reducing the capsule SG to less than 1.

While incorporating an effervescent material in the capsule device is an effective method of controlling the SG in the GI tract, there have been some limitations in this approach as most effervescent acid and base combinations do not generate an acceptable balloon inflation kinetics and temperature stability to make the capsule endoscope useful as a product to image the large intestine. FIG. 2 illustrates an example of balloon inflation kinetics for a capsule endoscope, where the balloon volume over time is shown. Once the capsule endoscope is swallowed and the protecting outer shell is disintegrated, the effervescent material inside the balloon comes in contact with moisture from the gastric fluid and as a result CO₂ (gas) is generated to expand the balloon volume. After the balloon is expanded to its maximum, the CO₂ (gas) pressure is at highest and begins to decrease at the time CO₂ (gas) diffuses through the balloon membrane wall faster than it is produced. At a certain balloon volume, the specific gravity (SG) of the capsule endoscope/balloon as a whole will become smaller than 1. “Specific Gravity” or SG means the ratio of the density of a body to the density of water at about physiologic temperature or about 37° Celsius. A body that is buoyant or floats on the surface of body liquid has its SG equal to about 1 or less than 1. A body that sinks in water has a SG greater than 1.

In FIG. 2, curve 210 represents the balloon volume vs time, where the time starts to being measured at the time the camera endoscope is swallowed by a patient. Line 220 indicates a threshold volume, where the SG is smaller than 1 when the balloon volume is over the threshold volume and the SG is larger than 1 when the balloon volume is below the threshold volume. As shown in FIG. 2, the time period, designated as rise time (Tx) refers to the period after the time that the capsule is swallowed to the time that the balloon volume reaches the threshold volume. The capsule endoscope will stay afloat for a period of time, designated as afloat time (Ta), until the balloon volume decreases to below the threshold volume. At this time, the capsule endoscope will sink in the body liquid. Ttb is the time from the capsule is ingested until the capsule is no longer buoyant, which is the same as the combined times of Tx and Ta.

In the field, various effervescent materials have been known for gas generation. For example, sodium bicarbonate, NaHCO₃ has been identified as a CO₂ containing material useful to control the balloon inflation kinetics in a desirable way. However, the standard grinded effervescent material, such as NaHCO₃, has limited thermally stability at 40° C. and even less at higher temperatures. Table 1 shows an example of inflation kinetics in water at 37° C. for an effervescent formulation based on NaHCO₃ and citric acid, where the rise time (Tx) to a volume where the capsule SG is −1, the afloat time Ta for the capsules having SG<1, and the combined time of Tx and Ta is referred as Ttb. Table 1 includes capsule balloons (using Balloon A) exposed to 25, 40, and 50° C. respectively for at least 24 hours prior to starting the balloon inflation experiments. As shown in Table 1 the Max Volume for the capsule balloons exposed to higher temperatures (40° and 50° C.) for an extended period of time are significantly lower than the Max Volume for capsule balloons stored at room temperature (25° C.) indicating that the effervescent material was degraded during exposure to higher temperatures

In addition, the Tx and Ta, vary substantially between the typical room temperature of 25° C. and the raised temperatures at 40° and 50° C. to the extent that the time to reach buoyancy (Tx) and time of staying afloat (Ta) are no longer clinically useful. As observed, capsule endoscopes formulated with standard grinded effervescent powder based on NaHCO₃ are not stable at elevated temperatures. For example, the average Ta is reduced from 9.45 hours to 5.11 hours, which may cause the capsule endoscope to have a difficult time to rise and pass through the ascending colon. For this reason, the capsule endoscopes formulated with standard grinded effervescent powder based on NaHCO₃ can typically not be conveniently shipped to hotter climates.

TABLE 1

| | Max Volume (mL) | | | Tx (hour) | | | Ttb (hour) | | | Ta (hour) | | |
|---|---|---|---|---|---|---|---|---|---|---|---|---|
| | Avg. | Std. Var | P. from RT | Avg. | Std. Var | P. from RT | Avg. | Std. Var | P. from RT | Avg. | Std. Var | P. from RT |
| RT (25° C.) | 1.69 | 0.01 | | 1.86 | 0.03 | | 11.31 | 0.51 | | 9.45 | 0.71 | |
| 40° C. | 1.48 | 0.03 | 0.026 | 2.53 | 0.19 | 0.012 | 12.03 | 3.01 | 0.378 | 9.50 | 3.20 | 0.952 |
| 50° C. | 1.23 | 0.01 | 1.18E−05 | 3.52 | 0.62 | 0.004 | 8.63 | 3.11 | 0.013 | 5.11 | 6.14 | 0.006 |

| | | | | |
|---|---|---|---|---|
| 40° C. vs 50° C. P value | 0.012 | 0.023 | 0.007 | 0.006 |

Accordingly, it is desirable to develop a capsule endoscope that contains thermally stable effervescent material for inflation control.

### BRIEF SUMMARY OF THE INVENTION

A capsule device with thermally-stable specific gravity control is disclosed. The capsule device comprises a capsule unit adapted to be swallowable by a human subject and an inflatable balloon comprising a thermally-stable effervescent formulation inside the inflatable balloon, where the thermally-stable effervescent formulation has a particular particle size between a first mesh size and a second mesh size, and the inflatable balloon is attached to the capsule unit. After the capsule unit with the inflatable balloon attached is swallowed, the inflatable balloon starts to inflate so as to lower specific gravity of a combination of the capsule unit and the inflatable balloon when the inflatable balloon is exposed to body fluid and moisture from the body fluid gets in touch with the thermally-stable effervescent formulation inside the inflatable balloon.

In one embodiment, the thermally-stable effervescent formulation comprises a CO₂ (gas) generating base such as sodium bicarbonate, sodium carbonate, potassium bicarbonate, potassium carbonate or any combination of these bases. In yet another embodiment, the thermally-stable effervescent formulation further comprises an acid. The acid selected may be crystalline or semi-crystalline, anhydrous, low or high molecular weight, and water soluble. In another example, the acid belongs to a group comprising citric acid, tartaric acids or monocalciumphosphate (Ca(H2PO4)2) and have an acid to base functional group stoichiometric ratio of close to 1 or above.

In one embodiment, the first mesh size is equal to 12 and the second mesh size is equal to 14. In another embodiment, the first mesh size is equal to 10 and the second mesh size is equal to 12. In yet another embodiment, the first mesh size is equal to 10 and the second mesh size is equal to 14. In yet another embodiment, the first mesh size is equal to 14 and the second mesh size is equal to 16. In yet another embodiment, the first mesh size is equal to 14 and the second mesh size is equal to 18.

### BRIEF DESCRIPTION OF THE DRAWINGS

FIG. 1 shows schematically a capsule camera system in the GI tract, where archival memory is used to store captured images to be analyzed and/or examined.

FIG. 2 illustrates an example of balloon inflation kinetics for a capsule endoscope, where the balloon volume vs time is shown.

FIG. 3 depicts the balloon inflation volume over time for a set of capsules incorporating an embodiment of the present invention exposed to higher temperature (60° C.) for 28 days and another set of baseline capsules (not exposed to 60° C. for 28 days), respectively.

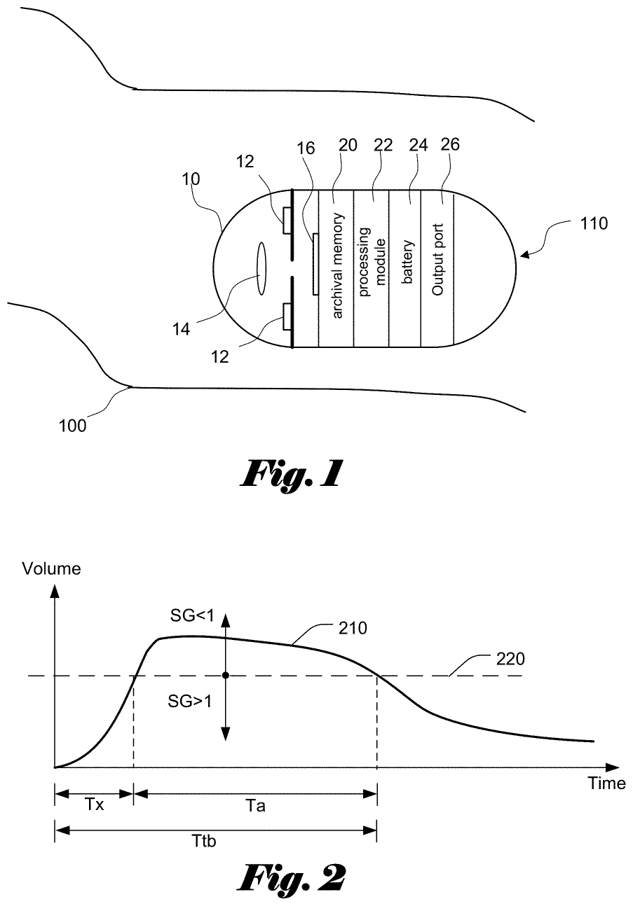

### DETAILED DESCRIPTION OF THE INVENTION

It will be readily understood that the components of the present invention, as generally described and illustrated in the figures herein, may be arranged and designed in a wide variety of different configurations. Thus, the following more detailed description of the embodiments of the systems and methods of the present invention, as represented in the figures, is not intended to limit the scope of the invention, as claimed, but is merely representative of selected embodiments of the invention.

Reference throughout this specification to “one embodiment,” “an embodiment,” or similar language means that a particular feature, structure, or characteristic described in connection with the embodiment may be included in at least one embodiment of the present invention. Thus, appearances of the phrases “in one embodiment” or “in an embodiment” in various places throughout this specification are not necessarily all referring to the same embodiment.

Furthermore, the described features, structures, or characteristics may be combined in any suitable manner in one or more embodiments. One skilled in the relevant art will recognize, however, that the invention can be practiced without one or more of the specific details, or with other methods, components, etc. In other instances, well-known structures, or operations are not shown or described in detail to avoid obscuring aspects of the invention.

The illustrated embodiments of the invention will be best understood by reference to the drawings, wherein like parts are designated by like numerals throughout. The following description is intended only by way of example, and simply illustrates certain selected embodiments of apparatus and methods that are consistent with the invention as claimed herein.

This present invention discloses a capsule device with an inflatable balloon unit including an effervescent material that is stable to at least 60° C. for at least 28 days and which has a shelf-life of at least one year even after the capsule has been exposed to 60° C. for an extended period of time.

In the present invention, effervescent materials with specific particle sizes are used as candidates for thermal stability as well as satisfactory mono-modal gas inflation behavior. Typically, the effervescent materials for gas generation are in a powder form. The stability of the effervescent material in the powder form may be more susceptible to higher temperatures. Accordingly, the present invention selects effervescent materials with specific particle sizes to achieve the goal of thermal stability. The type and amount of effervescent for a target capsule depend on the particular capsule design as well as the balloon membrane material and thickness. Some balloon design considerations are disclosed in U.S. Pat. No. 10,098,526, which is assigned to the same assignee as the present invention. According to the present invention, a specific particle size or sizes are selected for the effervescent material to achieve the thermal stability. In one embodiment, NaHCO₃ (sodium bicarbonate) is selected as the base (or CO₂ donor) of the effervescent material for a target capsule design. The selected particle size is 12-14 mesh. In other words, the effervescent particles can pass through mesh size 12, but will not pass mesh size 14. For mesh size 12, the screen opening size is about 1.68 mm (millimeter). For mesh size 14, the screen opening size is about 1.41 mm. In other words, the effervescent material selected to assemble a thermally stable capsule endoscope during storage/transportation needs to have specific particle size.

Bench tests were conducted by aging capsules with a balloon containing the effervescent material in particle form at mesh size 12-14 at 60° C. for 28 days (which corresponds to 1 year at room temperature). Six samples (n=6) of the capsules embodying the present invention and six baseline capsules (n=6) were used in this study. The total volume and inflation kinetics for the capsules embodying the present invention are compared to the baseline capsules, Table 2. The baseline capsules are reference capsules that were stored at room temperature, i.e., not exposed to 60° C. for 28 days. All capsule devices were suspended from an arm coupled to a weighing scale. The arm overhung a basin of water heated to about 37° C. Ballast was attached to the arm. The weight of the arm and ballast was zeroed out using a weighing scale software. Next, the capsule devices were attached to the arms with the ballasts and dropped into the water. The change in weight of the capsule devices were then recorded over the next 25 hours. As moisture entered the balloons the effervescent material inside the balloons was mobilized so the CO₂ (g) generating reaction could take place allowing the SG of the capsule devices to change: the SG decreased (as CO₂ (gas) produced from the reaction between the acid and the base of the effervescent), the SG reached a minimum value (corresponding to a 100% inflation condition), and then the SG increased as CO₂ (gas) diffused out from the balloons.

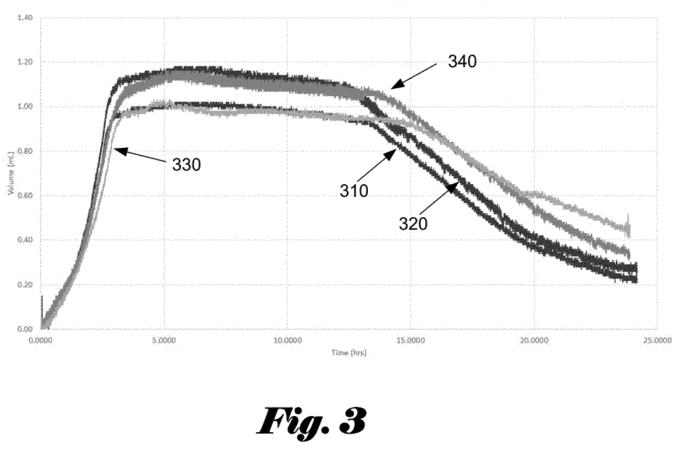

The graph in FIG. 3 depicts the balloon inflation volume over time for a set of capsules incorporating an embodiment of the present invention exposed to higher temperature (60° C.) for 28 days and another set of baseline capsules (not exposed to 60° C. for 28 days), respectively. In order to visually distinguish the curves easily, only two sets of curves are shown in FIG. 3. Inflation volumes above about 0.9 mL for these capsule devices correspond to SG<1. While inflation volumes below 0.9 mL correspond to SG>1. In FIG. 3, the dark-colored curves (310, 320) represent typical inflation kinetics of the baseline capsule, and the light-colored curves (330, 340) represent typical inflation kinetics of the capsule incorporating an embodiment of the present invention. Based on this data and the data in Table 2, it has been demonstrated that an effervescent material with larger particles (12-14 mesh) makes the capsule endoscope thermally stable at temperatures up to 60° C. As shown in FIG. 3 and Table 2, the capsule balloons (using Balloon B) incorporating an embodiment of the present invention being exposed to higher temperature (e.g., 60° C. for 28 days) still generates the same max volume and total volume (area under the curve). In addition, the capsule incorporating an embodiment of the present invention being exposed to higher temperature (e.g., 60° C. for 28 days) exhibits about the same inflation kinetics as the baseline capsule not exposed to 60° C. for 28 days, FIG. 3 and Table 2. For example, the rise time Tx for the capsule to reach SG˜1 (i. e., the volume reaching about 0.9 mL) is about the same for the baseline capsule and the capsule exposed 60° C. for 28 days incorporating an embodiment of the present invention. Also, the capsules incorporating an embodiment of the present invention 60° C. for 28 days stay afloat for about the same period of time as the baseline capsules not exposed to 60° C. for 28 days.

Most importantly, the max volume for the capsule balloons (using Balloon B) incorporating the effervescent material according to the present invention and exposed to 60° C. for 28 days is about 1.09 mL, which is similar to the max volume (1.11 mL) for the baseline capsule balloons (not exposed to 60° C. for 28 days), Table 2. On the other hand, as outlined in Table 1 the max volume of a capsule balloon (using Balloon B) with the conventional effervescent material (i.e., in powder form) which is not stable to increased temperatures would suffer a max volume drop of 27.2% (from 1.69 mL to 1.23 mL) even at a temperature of 50° C. The nominal size of Balloon A (1.69 mL) is larger than the nominal size of Balloon B (˜1.11 mL).

TABLE 2

| | Max Volume (mL) | | Tx (hour) | | Ttb (hour) | | Ta (hour) | |
|---|---|---|---|---|---|---|---|---|
| | Avg. | Std. Var. | Avg. | Std. Var. | Avg. | Std. Var. | Avg. | Std. Var. |
| RT (25° C.) | 1.11 | 0.07 | 2.95 | 0.4 | 12.02 | 1.26 | 9.07 | 1.25 |
| 60° C. for 28 days | 1.09 | 0.06 | 3.27 | 0.59 | 13.50 | 2.24 | 10.23 | 2.81 |

While FIG. 3 illustrates an example of a thermally stable capsule endoscope using effervescent formulation according to an embodiment at 60° C., the thermal stability is achieved for all temperatures up to 60° C.

While the particular effervescent material comprising NaHCO₃ as the base is selected to demonstrate the thermally stable characteristics, other effervescent materials may also be used to practice the present invention. For example, besides sodium bicarbonate (NaHCO₃), other effervescent materials such as potassium bicarbonate, sodium carbonate, potassium carbonate, or any mixture of these. Furthermore, the effervescent formulation typically comprises an acid to achieve a desire inflation kinetics. The acid selected may be crystalline or semi-crystalline, anhydrous, low or high molecular weight, and water soluble. For example, the acid belongs to a group comprising citric acid, tartaric acids or monocalciumphosphate (Ca(H₂PO₄)₂) and should have an acid to base functional group stoichiometric ratio of close to 1 or above.

The definition of functional group stoichiometric ratio is for example when you mix citric acid and sodium bicarbonate the stoichiometric ratio for the reaction is:

H3C6H5O7(aq)+3NaHCO3(aq)→Na3C6H5O7(aq)+3H2O(l)+3CO2(gas) (I)

but since one citric acid molecule have three functional groups (—COOH)) it can donate protons to three sodium bicarbonate molecules. Thus as written in equation (I) the functional group stoichiometric ratio is 1. The reaction rate can be increased by adding excess of citric acid and still generate the same volume CO2 (gas), while if you reduce the amount of citric acid not only will the CO2 (gas) generation rate be reduced but the total volume of CO2 (gas) generated will also be less. If the excess of citric acid is significant the CO2 (gas) generation rate will also be significantly higher.

While the particle size of mesh 12-14 is selected for NaHCO₃, other particle size or sizes may be required for other effervescent materials. For example, a larger particle size of mesh 10-12 may be selected to achieve a desired inflation kinetics for a particular capsule design. In another embodiment, a larger particle sizes of mesh 10-14 may be selected to achieve a desired inflation kinetics for a particular capsule design. In another example, a smaller particle size of mesh 14-16 may be selected to achieve a desired inflation kinetics for a particular capsule design. In yet another embodiment, a smaller particle sizes of mesh 14-18 may be selected to achieve a desired inflation kinetics for a particular capsule design.

The invention may be embodied in other specific forms without departing from its spirit or essential characteristics. The described examples are to be considered in all respects only as illustrative and not restrictive. The scope of the invention is, therefore, indicated by the appended claims rather than by the foregoing description. All changes which come within the meaning and range of equivalency of the claims are to be embraced within their scope.

1. A capsule device, comprising:
a capsule unit adapted to be swallowable by a human subject; and
an inflatable balloon comprising a thermally-stable effervescent formulation inside the inflatable balloon, wherein the thermally-stable effervescent formulation has a particular particle size between a first mesh size and a second mesh size, and wherein the inflatable balloon is attached to the capsule unit; and
wherein after the capsule unit with the inflatable balloon attached is swallowed, the inflatable balloon starts to inflate so as to lower specific gravity of a combination of the capsule unit and the inflatable balloon when the inflatable balloon is exposed to body fluid and moisture from the body fluid gets in touch with the thermally-stable effervescent formulation inside the inflatable balloon.

2. The capsule device of claim 1, wherein the thermally-stable effervescent formulation comprises sodium bicarbonate, potassium bicarbonate, sodium carbonate, potassium carbonate or any combination of these.

3. The capsule device of claim 2, wherein the thermally-stable effervescent formulation further comprises an acid.

4. The capsule device of claim 3, wherein the acid is selected from an acid group comprising crystalline, semi-crystalline, anhydrous, low or high molecular weight, and water soluble.

5. The capsule device of claim 2, wherein the thermally-stable effervescent formulation further comprises an excess acid.

6. The capsule device of claim 5, wherein the excess acid belongs to an excess acid group comprising citric acid, tartaric acids or monocalciumphosphate (Ca(H₂PO₄)₂).

7. The capsule device of claim 6, wherein the excess acid has an acid to base functional group stoichiometric ratio of close to 1 or above.

8. The capsule device of claim 2, wherein the first mesh size is equal to 12 and the second mesh size is equal to 14.

9. The capsule device of claim 2, wherein the first mesh size is equal to 10 and the second mesh size is equal to 12.

10. The capsule device of claim 2, wherein the first mesh size is equal to 10 and the second mesh size is equal to 14.

11. The capsule device of claim 2, wherein the first mesh size is equal to 14 and the second mesh size is equal to 16.

12. The capsule device of claim 2, wherein the first mesh size is equal to 14 and the second mesh size is equal to 18.

13. The capsule device of claim 1, wherein the thermally-stable effervescent formulation comprises sodium bicarbonate.

14. The capsule device of claim 13, wherein the thermally-stable effervescent formulation further comprises an acid.

15. The capsule device of claim 14, wherein the thermally-stable effervescent formulation further comprises an excess acid.

16. The capsule device of claim 15, wherein the first mesh size is equal to 12 and the second mesh size is equal to 14.

17. The capsule device of claim 15, wherein the first mesh size is equal to 10 and the second mesh size is equal to 12.

18. The capsule device of claim 15, wherein the first mesh size is equal to 10 and the second mesh size is equal to 14.

19. The capsule device of claim 15, wherein the first mesh size is equal to 14 and the second mesh size is equal to 16.

20. The capsule device of claim 15, wherein the first mesh size is equal to 14 and the second mesh size is equal to 18.
